(19) United States
(12) Patent Application Publication
(10) Pub. No.: US 2008/0154251 A1
(43) Pub. Date: Jun. 26, 2008

(54) Interchangeable Tips for Medical Laser Treatments and Methods for Using Same

(75) Inventors: B. Wayne Stuart, Santa Cruz, CA (US); Leonard C. DeBenedictis, Palo Alto, CA (US); Christopher B. White, San Jose, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 11/777,196

(22) Filed: Jul. 12, 2007

Related U.S. Application Data
(63) Continuation-in-part of application No. 11/223,787, filed on Sep. 8, 2005.
(60) Provisional application No. 60/609,037, filed on Sep. 9, 2004.
(60) Provisional application No. 60/807,333, filed on Jul. 13, 2006.

(51) Int. Cl. A61B 18/20

(52) U.S. Cl. 606/13

(57) ABSTRACT

A typical treatment system for use with tip embodiments includes an optical energy source, such as, for example, a laser. A set of tips may be interchangeably attached to the treatment system, for example to alter the system parameters and the treatment provided through the individual tips. Embodiments of the present invention include tips with a security chip and/or a memory. A security chip protects the treatment system from use with unauthorized tips, and the memory stores information about the tips and/or the treatment system to enhance the treatment

### RELATED APPLICATIONS

This application (a) is a continuation-in-part of pending U.S. patent application Ser. No. 11/223,787, “Interchangeable Tips for Medical Laser Treatments and Methods for Using Same,” filed Sep. 8, 2005 by Len DeBenedictis et al.; which claims priority under 35 U.S.C. § 119(e) to U.S. Provisional Patent Application Ser. No. 60/609,037, “Interchangeable Tips for Medical Laser Treatments and Methods for Using Same,” filed Sep. 9, 2004 by Len DeBenedictis et al.; and (b) claims priority under 35 U.S.C. § 119(e) to U.S. Provisional Patent Application Ser. No. 60/807,333, “Interchangeable Tips for Medical Laser Treatments and Methods for Using Same,” filed Jul. 13, 2006 by Len DeBenedictis et al. The subject matter of the foregoing is incorporated herein by reference in its entirety.

### FIELD OF THE INVENTION

The present invention relates generally to methods and apparatus for providing medical or surgical treatment using optical energy, and in particular to interchangeable tips coupled to a handpiece in a treatment system and a method for using such tips for treatment of tissue (e.g., human skin) using optical radiation.

### BACKGROUND OF THE INVENTION

Lasers and other intense light sources are used for various types of tissue treatment, including dermatological tissue treatment. Optical energy, particularly laser energy, is commonly used as a versatile tool in medicine to achieve desired outcomes in the tissue that is treated. For example, lasers have been used to treat common dermatological problems such as hypervascular lesions, pigmented lesions, acne scars, rosacea, hair removal, etc. Additionally, lasers are also used in aesthetic surgery for achieving better cosmetic appearance by resurfacing the skin and remodeling the different layers of skin to improve the appearance of wrinkled or aged skin. Generally, skin resurfacing is understood to be the process by which the top layers of the skin are completely removed by using chemicals, mechanical abrasion or lasers to promote the development of new, more youthful looking skin and stimulate the generation and growth of new skin. In laser skin remodeling, laser energy penetrates into the deeper layers of the skin and is aimed at stimulating the generation of and/or altering the structure of extra-cellular matrix materials, such as collagen, that contribute to the youthful appearance to skin.

During dermatological tissue treatment utilizing light, a light beam irradiates the skin surface of a patient. Generally, lasers that are used for such treatment operate at a wavelength that is absorbed by one of the natural chromophores in the skin, such as water, although chromophores may also be added to the tissue. In the case of water as the primary chromophore, cellular and interstitial water absorbs light energy and transforms the light energy into thermal energy. The transport of thermal energy in tissues during treatment is a complex process involving conduction, convection, radiation, metabolism, evaporation and phase change that vary with the operational parameters of the light beam. It is important in such procedures not to damage tissue underlying or surrounding the target tissue area. If the light beam optical operational parameters, such as wavelength, power, the intensity of the light, pulse duration, rate of emission, etc. are properly selected, cellular and interstitial water in the patient's skin is heated causing temperature increases that produce a desired dermatological effect. Conversely, improper selection of the optical operational parameters can result in undertreatment or overtreatment of the tissue. Therefore, it is desirable to accurately control optical operational parameters used in the treatment so that the light is delivered to the tissue with the proper fluence and in a uniform, controllable manner.

Devices for dermatological tissue treatment include a hand-held delivery apparatus, sometimes referred to as a handpiece. A handpiece is a preferred means by which physicians apply treatment to tissue. During treatment, the handpiece emitting light is moved by a physician's hand along the tissue to be treated. Treatment level from such a device is typically set in advance by manually selecting the light beam operational parameters. The operational parameters, which for example include power level, energy, pulsation rate, temperature, light intensity, and current, determine the degree of treatment of the entire treatment process.

A typical approach of conventional handpieces is to produce a macroscopic, pulsed treatment beam that is manually moved from one area of the skin to another in a patchwork like manner in order to treat a larger region of skin tissue. Such an approach has the disadvantage of producing artifacts and sharp boundaries associated with the inaccurate positioning of the individual treatments with respect to the treated skin surface.

Another disadvantage of conventional handpieces is that, as discussed above, the laser operational parameters defining the selected level of treatment are typically pre-set once for the entire course of treatment. The individual tissue properties of each patient are factored in based on a preliminary tissue assessment prior to the treatment and the treatment can proceed using the predetermined operational parameters.

For example, some handpiece apparatuses may provide feedback indicating to the physician the rate of the handpiece movement which allows the physician to adjust the treatment speed. But this handpiece apparatus requires the physician to treat at a pre-selected rate of motion. The disadvantage of this apparatus is that it restricts the physician to a single treatment speed. In large flat areas, such as the cheek, it is desirable to treat at a high speed. In highly contoured areas, such as the lip, it is desirable to treat at a lower speed. Restricting the physician to a pre-selected rate of motion limits the flexibility of the physician when treating regions, such as the face, that include both large flat areas and highly contoured areas that are in close proximity. Additionally, if the speed of the handpiece changes during the treatment procedure, the apparatus does not provide for automatic adjustment of its operational parameters to compensate for the changed rate of movement, leading to uneven treatment.

The application of robotic means used in the field of dermatological or cosmetic surgery could overcome the limitation of human imprecision. However, one disadvantage of typical conventional robotic apparatuses is that they lack the necessary direction and judgment in treatment that a physician provides. Although robotics is precise, it is not typically intelligent enough to make complex choices or react to unforeseen circumstances during treatment. Additionally, robots deprive a physician of discretion in an aesthetic sense. Another disadvantage of the typical conventional robotic apparatus is that the full treatment may require complete immobilization of the patient. Alternatively, a sophisticated image stabilization system must be employed to compensate for patient's movement.

Many current medical laser systems are used in contact with tissue being treated. Such contact systems require cleaning and special care to maintain cleanliness, if not sterility depending on the treatment, as well as efficacy. Such contact systems often include a window or some aperture through which energy passes. If such windows or apertures become blocked—such as by foreign substances, scratches, chips, or cracks—then the device typically will not function properly. Conventional systems typically have a monolithic handpiece with unchangeable mechanical, electrical and optical components, configurations and connections.

The present invention provides apparatuses and methods which significantly reduce the problems associated with the existing medical laser systems and methods.

### SUMMARY OF THE INVENTION

In general, the present invention features an interchangeable tip for a medical treatment system. A typical treatment system for use with tip embodiments includes an electromagnetic energy source, such as, for example, a laser or radio frequency generator. A set of tips may be interchangeably attached to the treatment system, for example to alter the system parameters and the treatment provided through the individual tips. Embodiments of the present invention include tips with an attached security chip and/or a memory. A security chip protects the treatment system from use with unauthorized tips, and the memory stores information about the tips and/or the treatment system to enhance the treatment. The tips may be authenticated through the use of a keyed hash algorithm, such as, for example, the SHA-1 algorithm. The authentication may occur through a communications device, such as an electrical connector or wireless connection, attached to the tip.

Embodiments of the present invention feature a removable tip apparatus for use with a medical light energy treatment system that includes a handpiece. The tip apparatus includes a housing, a light energy pathway and a security chip. The housing is shaped so that the tip can be removably attached to the distal end of the handpiece. When the tip is attached, the light energy pathway provides a path for transmitting light energy from the distal end of the handpiece through the tip apparatus to a target area. The security chip is used to determine whether or not to enable delivery of light energy to the distal end of the handpiece. For example, if the security chip is invalid or missing, then light energy may be disabled. If the security chip is valid, indicating that the tip is authentic, then the light energy may be transmitted.

In another aspect of the present invention, a medical light energy treatment system includes a light source, a handpiece and a set of two or more different but interchangeable tips. The handpiece is optically coupled to the light source and is configured to transmit light energy from the light source to a distal end of the handpiece. The tips can be removably attached to the distal end of the handpiece. When attached, each tip transmits at least a portion of the light energy from the distal end of the handpiece through the tip to a target area. However, the tips are different. For example, the tips may have different configurations, different physical designs or dimensions and/or different operating characteristics. The differences operate to cause different treatments.

In a further embodiment, a medical light energy treatment system includes a light source, handpiece, interchangeable tips and a host processor. The light source, handpiece and tips operate similarly as above. However, the tips also include attached memory that stores tip-specific data, for example system usage time for the tip, energy transmitted through the tip, energy pulse count data for the tip, tip type, tip configuration and/or tip parameters. The host processor, which is located external to the tips, communicates with the memory to access the tip-specific data as part of the treatment process.

Other aspects of the invention include methods, systems and applications relating to the embodiments described above

### BRIEF DESCRIPTION OF THE DRAWINGS

These and other features, objects and advantages of the present invention are more readily understood from the following detailed description in conjunction with the accompanying drawings, where:

FIG. 1 is an illustration of a medical treatment system including a handpiece.

FIG. 2 is a diagrammatic view of an apparatus showing feedback control of the laser power for controlled tissue treatment.

FIG. 3 is a side view of a handpiece including a detector and an optical element.

FIG. 4 shows a detector of the handpiece shown in FIG. 3 in sensing mode in greater detail

FIG. 5 shows a tip according to one embodiment of the present invention.

FIG. 6 shows a tip according to an alternate embodiment of the present invention.

FIG. 7 illustrates various dimensions and configurations of a tip according to embodiments of the present invention.

FIGS. 8a and 8b illustrate embodiments of the present invention with different tip dimensions and the impact of such difference on treatment parameters.

### DETAILED DESCRIPTION

Much of the discussion and many of the embodiments discussed herein relate to dermatology applications. However, this should not be viewed as limiting the inventions herein solely to dermatology. Generally, treatment of biological tissues by electromagnetic energy through interchangeable tips is included in the present invention. While the embodiments will focus on the use of optical energy, the scope of the invention is intended to extend to electromagnetic energy such as radio frequency or microwave treatment devices as well. Preferred radio-frequency and microwave treatment devices will have an electromagnetic frequency within the range of 300 kHz to 3 GHz and more preferably within the range of 4 MHz to 10 MHz. The electromagnetic frequency can be chosen based on the desired depth of penetration of the energy.

Embodiments of the present invention include tips that are interchangeable and may be disposable. Additional benefits include the ability to change treatment system parameters and treatment parameters by changing tips, which is typically much simpler and more efficient than changing optical elements or the mechanical configuration of the handpiece itself. For example, different tips may have different dimensions along the optical axis of the tip in order to alter the focal depth of the treatment beam into the tissue and/or the spot size of the treatment beam at the tissue surface. Different tips may have different sizes in dimensions other than those parallel to the optical axis of the treatment beam. Different tips may also have different shapes, some of which may be sized and shaped to treat specific conditions or specific anatomical structures, for example. Different tips may have different filter properties in order to limit the wavelength(s) of the light transmitted through the tip. Further alternate embodiments may include different LEDs for various tracking or sensing purposes, as well as varying the location of the LED or the window through which the LED light is transmitted to the tissue surface. By keeping the optical system, laser system and handpiece mechanics in a relatively constant configuration in the handpiece and changing tips, cost and complexity is reduced while providing a variety of effective treatment parameters from a single handpiece.

Further embodiments of the present invention include interactions between the tips and the medical laser system to which the tip is attached. Embodiments of the present invention will typically include tips that have an attached security chip and/or a memory. The memory will often be part of a security chip, although separate or additional memory may also be included. The memory may hold various codes and/or data. For example, an encrypted security code may be included to ensure that only approved and/or specified tips are used with a given medical laser system. Additionally, information relating to the usage of the tip or the system may be stored in the memory. For example, such tip usage data may include: accumulated time of use; clock time since the tip was attached to the medical laser system or the medical laser system was first turned on with the tip attached; number of pulses transmitted through the tip; accumulated energy or fluence transmitted through the tip; accumulated power transmitted through the tip; number of patients treated using the tip; number of treatment spots laid down through the tip; etc. Further alternate embodiments may include storing tip and/or treatment system information in the memory. For example, the tip type may be stored in the memory so that the treatment system controller or processor can read the tip type and set system operating parameters accordingly. For example, the tip type information may include tip dimensions such as the dimension along the optical axis of the tip so that the treatment system can calculate the spot size at the tissue surface and/or the focal depth of a treatment beam in the tissue. Using these calculations, the treatment system may, for example, limit the maximum energy applied per pulse in order to control damage to the tissue. Alternately, tip information may include the dimensions of the treatment area for the tip, and the system may then alert the system user to these dimensions and the resultant impact on the treatment regimen to be used with that tip. Individual tips in a set or plurality of tips configured to be attached to a single system or handpiece may be designed for specific purposes such as, for example: treatment of wrinkles, scars, pigmented lesions, hair removal or growth; drug delivery; treatments around eyes, neck, nose; targeting of anatomical structures, such as veins or lesions; etc. A user interface attached to the treatment system allows a user to obtain information relating to the tip as well as information relating to a given tip's impact on the treatment system configuration and treatment parameters. A user may then alter treatment parameters and/or switch tips to achieve the desired treatment.

Embodiments of improved laser treatment systems and methods employing robotics and motion control feedback are found at co-pending U.S. patent applications Ser. Nos. 10/745,761 and 60/605,092 (Attorney docket number 23920-09449) each assigned to Reliant Technologies, Inc., and entitled “Method And Apparatus For Monitoring And Controlling Laser-Induced Tissue Treatment”, filed on Dec. 23, 2003, and Aug. 26, 2004, respectively, and both incorporated herein by reference in their entireties. These embodiments typically use a light-emitting diode (LED) or some other illumination source to illuminate the tissue so that it may be more easily detected. Further, co-pending U.S. application Ser. No. 10/367,582, entitled “Method And Apparatus For Treating Skin Using Patterns Of Optical Energy”, filed on Feb. 14, 2003, and co-pending U.S. application Ser. No. 10/888,356 (Attorney docket number 23920-09289), entitled “Method And Apparatus For Fractional Photo Therapy Of Skin”, filed on Jul. 9, 2004, each assigned to Reliant Technologies, Inc., describe the use of fractional laser therapy and the value of discrete microscopic treatment zones with untreated tissue left between such zones, both of which applications are incorporated herein by reference in their entireties.

In laser treatments including those using microscopic spots (i.e. typically less than about 500 microns in diameter, and preferably less than about 200 microns, typically measured at the largest necrotic zone lesion dimension perpendicular to the optical axis of the treatment beam), various parameters are important to producing the desired and effective treatment results. For example, important parameters may include one or more of the following: the wavelength of the light transmitted to the tissue; the size of the treatment spot at the tissue surface; the focal depth of the treatment beam, typically measured from the tissue surface or the surface of the contact surface of the handpiece; the amount of heating or cooling at the tissue surface; and the configuration of the exit window or aperture for the treatment handpiece (e.g., contact or non-contact windows, window or aperture shape), etc. Embodiments of the present invention include tips that enhance the effectiveness of the inventions and embodiments described in the above-referenced co-pending patent applications. However, tip embodiments described herein are not limited solely to use in conjunction with the afore-mentioned patent applications or the inventions described therein.

Treatment systems that use microscopic spot sizes or focus to depths of less than 4 mm into the skin typically use higher treatment fluences of optical energy than systems with larger spot sizes. The higher fluences can cause damage to the tip window and cause significant scattering, which is also more significant for microscopic spot sizes and may cause undertreatment or inconsistent treatment. Therefore, it is desirable to have an apparatus for storing tip usage data securely so that the system can verify that tips are replaced after a defined amount of usage. The tip usage data can be stored securely on the tip in the memory using the encryption algorithms described herein.

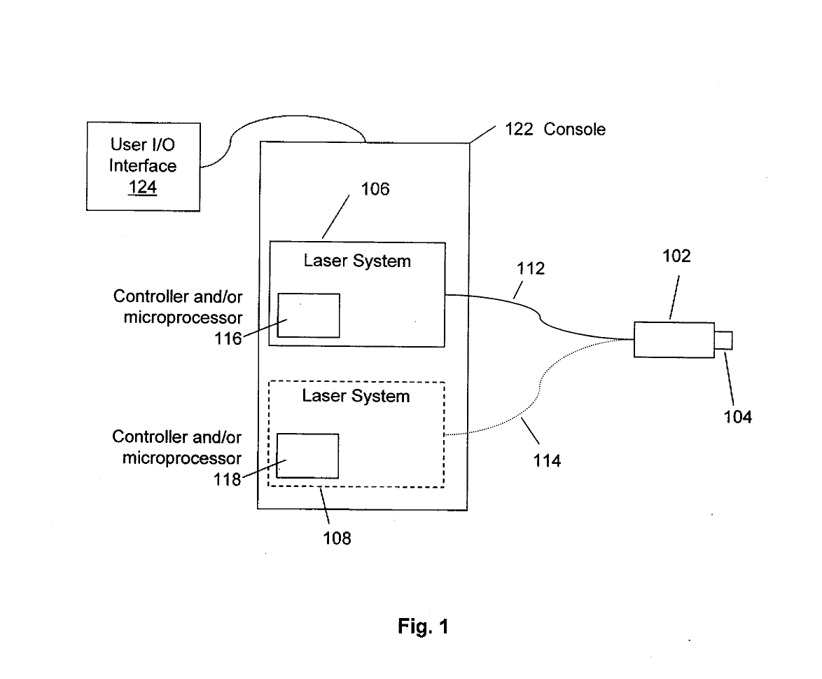

FIG. 1 shows the basic elements of a typical medical treatment system. In this treatment system, a handpiece 102 is coupled to a console 122 via a cable 112. Console 122 may not be included in all embodiments, in which case laser system(s) and controllers may be free-standing and separate with various electrical and optical connections there between. A handpiece is typically maneuverable and sized for manipulation by human hand. Cable 112 typically carries and protects various optical fibers and electrical wires. The console 122 typically holds one or more light systems, such as, for example, a laser system 106. The optical fibers in cable 112 are optically coupled to the one or more light systems and the handpiece 102 in order to transmit the light energy from the light system(s) to the handpiece. A user input/output (I/O) interface 124 is typically included in or attached to the treatment system so that a user may interact with, control and receive information from the treatment system.

FIG. 1 shows an example of a handpiece 102 connected to two laser systems 106 and 108. In the example shown in FIG. 1, laser systems are depicted as the light systems. However, one skilled in the art will recognize that a non-laser light source, such as flashlamps or LEDs, may be used in place of a laser. A second laser system 108 may be coupled to the handpiece 102. Optical pathways in cables 112 and/or 114 are typically used to transmit light from the laser system(s) 106 and/or 108 to the handpiece 102. Laser systems 106, 108 typically include a control system 116, 118 for the laser; the control system may be internal or external, and may be shared between lasers. The control system typically includes a controller, microprocessor and/or digital signal processor (DSP) along with software or firmware for use in controlling operational characteristics of the laser, such as, for example, pulse length, energy, wavelength (for tunable lasers), duty cycle, and so forth. One skilled in the art will recognize that two laser systems 106 and 108 may transmit light through a single optical fiber, and further a single laser may produce multiple wavelengths. Embodiments of the present invention contemplate and include these laser system configurations. In embodiments of the present invention, an interchangeable and disposable tip 104 is coupled to the treatment end of the handpiece 102. Embodiments of the tips in the present invention typically have an outer shell or skin formed from bio-safe plastics or metals, and may be hermetically sealed.

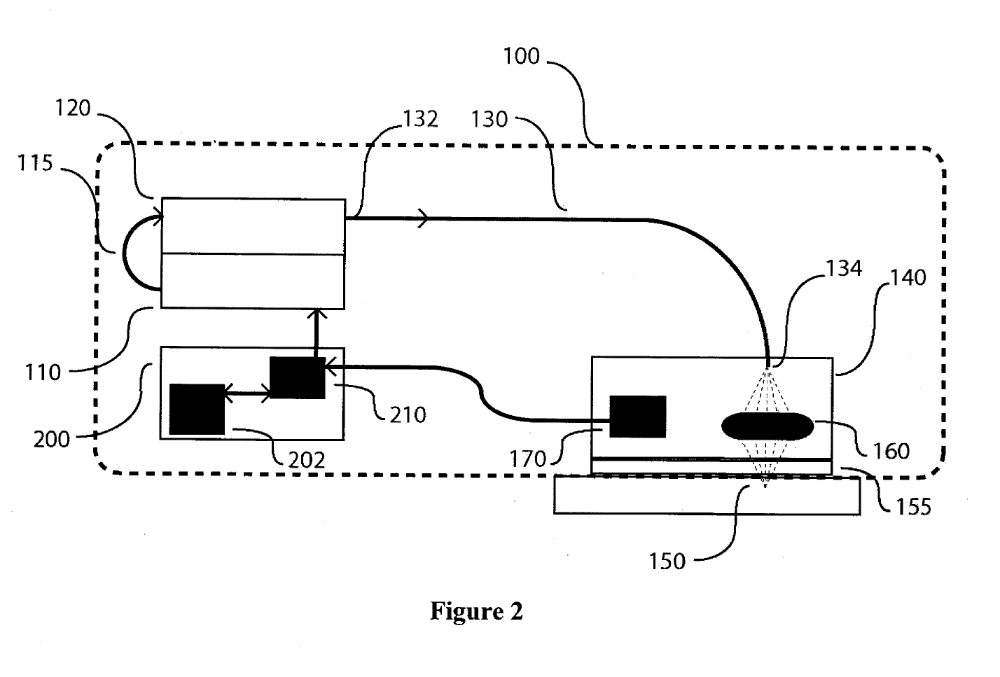

FIG. 2 illustrates in greater detail the laser treatment systems depicted in FIG. 1 above. FIG. 2 shows controlled tissue treatment system 100 in accordance with aspects of the present invention. In accordance with the example shown in FIG. 2, system 100 comprises power source 110 that energizes light emitter 120 for emission of a light beam via an electrical or optical connection 115; optical fiber 130 for transmission of the light beam; movable handpiece 140 with an interchangeable tip (not shown in detail here) and with an optical element 160 coupled to optical fiber 130 for emission of the light beam towards target area 150; detector 170 for detecting variations in positional parameters of handpiece 140; and controller 200 for controlling operational parameters of the light beam emitted towards target area 150 in response to the detected variations in the handpiece positional parameters. Light is typically passed through an optically transparent window 155 that may be flat or curved in one or more aspects. Optical element 160, detector 170, and window 155, in whole or in part, may be coupled to and/or enclosed within an interchangeable tip according to embodiments of the present invention. The connection 115 may consist of simply a free-space region through which an optical beam is passed. Controller 200 may comprise processor 202 for calculating new operational parameters and interface unit 210 for selecting and adjusting operational parameters of apparatus 200. The controller 200 may control operational parameters by adjusting parameters in at least one of the following: power source 110, light emitter 120, and optical element 160. For clarity, only one of these configurations is illustrated.

Light emitter 120 of treatment system 100 may be any optical power source. Light emitter 120 may be implemented, at least in part, using one or more light power sources. For certain applications, light emitter 120 may desirably include multiple light power sources arranged in an array, such as a one-dimensional array or two-dimensional array. It is preferred that the light power source utilized in the present invention is a laser, although other non-laser optical sources may be used. Suitable lasers according to the invention may include noble gas lasers (e.g., argon lasers, helium-neon lasers, etc.), diode lasers, fiber lasers, and tunable lasers. However, it must be understood that the selection of a particular laser for the tissue treatment system 100 is dependent on the type of the dermatological treatment selected for a particular application. Light emitter 120 is typically adapted to produce optical power between about 1 W and about 100 W, preferably about 30 W.

Light emitter 120 emits one or more optical beams having one or more wavelengths. In laser-induced tissue treatment, each optical beam may be characterized by a particular set of optical operational parameters that are selected to produce a desired dermatological effect on target area 150. Operational parameters of the light beam may include optical fluence, power, pulsation rate, duty cycle, light intensity, timing of pulse initiation, pulse duration, and wavelength.

In some embodiments, light emitter 120 is preferably capable of generating light at wavelengths with high absorption in water. Cellular water absorbs light energy and transforms the light energy into heat. Preferably, wavelengths larger than 190 nm, such as wavelengths in the range from 190 nm to 10600 nm, preferably from 700 nm to 1600 nm, and most preferably about 1550 nm are used in the apparatus 200. Desirably, light emitter 120 is an erbium-based fiber laser designed for about 1550 nm range operation. Light emitter 120 may be capable of providing one wavelength or a range of wavelengths or may be tunable across a range of wavelengths. One or more light emitters 120 may be powered by power source 110 to produce a variety of different wavelengths or wavelength ranges used in dermatological treatment. Light emitter 120 may be adapted to selectively produce pulses of laser light at a frequency of between 0 to about 50,000 pulses per second and preferably 0 to about 1,000 pulses per second. Preferably, light emitter 120 emits a beam having pulse energy per treatment spot of about 1 mJ to about 1000 mJ, more preferably between about 10 mJ and about 30 mJ, each pulse having a pulse duration per treatment spot between about 0.1 ms and about 30 ms, more preferably about 1 ms.

In some embodiments, power source 110 and light emitter 120 are typically used, for example, for non-ablative coagulation of an epidermal and/or a dermal layer of the target area 150. Typically, for this purpose, an optical fluence incident to target tissue area 150 greater than about 5 J/cm², such as an optical fluence in the range from about 10 J/cm² to about 1000 J/cm², is adequate for coagulating tissue. Generally, the optical fluence is adapted to the wavelength and the tissue to be treated. If various dermatological effects are desired, the power source 110 and light emitter 120 may be selected with the capacity to produce optical operational parameters suitable for other types of tissue treatment. For example, if ablation of an epidermal layer of the target area 150 is desired, the power source 110 and the light emitter 120 may be used with the capability to emit a light beam with a wavelength of about 2940 nm and optical fluence higher than 10 J/cm².

Optical fiber 130 may be any optical apparatus suitable for transmission of light emitted from light emitter 120. Fiber 130 may be constructed of a material that allows for free manipulation of the handpiece 140 and for repeated bending in order to direct the light beam from emitter 120 to various portions of target area 150. Fiber 130 may have beam-inlet end 132 that is aligned with the light beam emitted from light emitter 120 so that the light beam is coupled into optical fiber 130, and beam-outlet end 134 for emission of the transmitted light beam to handpiece 140. More than one fiber may be used to transmit the light beam from emitter 120 to handpiece 140. Alternatively, other optical delivery mechanisms 130, e.g., mirrors or waveguides may be used to guide the light beam from the light emitter 120 to the proximal end of handpiece 140.

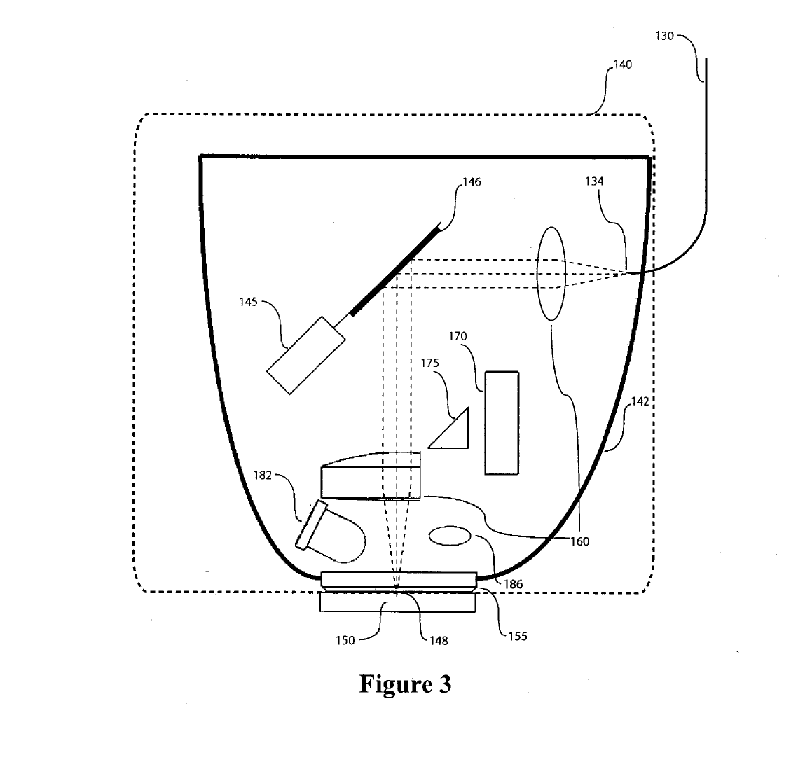

Referring to FIG. 3, like elements have similar reference numerals to those for FIG. 2. The housing 142 of handpiece 140 is a unit adapted for convenient holding by a human hand during the delivery of dermatological treatment. Shape of housing 142 of the handpiece 140 provides for a wide range of motion to manipulate the handpiece during treatment. Housing 142 may be made out of a light plastic, such as Kydex and may hold optics and electronics used for dermatological tissue treatment. Housing 142 may be connected to fibers 130 near the beam-outlet ends 134 and may contain a structure that allows the light beam to be guided through housing 142 and to be emitted from a tip (not shown in detail here) attached to the distal end of the housing, so that the light beam can propagate towards target area 150. For the most efficient treatment, it is preferred to direct and point the light beam emitted from output 148 at a substantially right angle to tissue 150.

Handpiece 140 may further include optical element 160 that is optically coupled to fibers 130. Optical element 160 directs optical energy from fibers 130 to target tissue area 150. In some embodiments, optical element 160 directs optical energy to target area 150 by focusing or collimating the light beams emitted from fibers 130 to one or more treatment zones within target area 150. Optical element(s) 160 may be implemented using one or more optical elements, such as mirrors, optical lenses, optical windows, rotating elements, counter-rotating wheel elements, electro-optic elements, acousto-optic elements, etc. Typically, for non-ablative treatment, the swath width of target area 150 is pre-selected at about 0.5 cm to about 2.0 cm.

Optical element(s) 160 may be configured to allow for control of the microscopic treatment patterns and density of the treatment zones. As will be discussed in greater detail below, substantially uniform pre-selected pattern and density of the treatment zones across the entire treated tissue area may be achieved by controlling optical element 160.

Handpiece 140 may further comprise deflector 146 or scanner mechanism. Deflector 146 may be an optical component suitable for deflecting the light beam of the wavelength pre-selected for the treatment, such as mirrors, prisms, grids, diffractive optical elements, holographic elements, rotating elements, etc. Deflector 146 may be operationally coupled to optical element 160 to modify the light beam emitted from optical element 160. Preferably, deflector 146 is movably mounted within housing 142 for displacement by actuator 145 in response to a controlling signal. Actuator 145 may be a piezoelectric, galvanometer, rotating element, etc., and operates to adjust the position of deflector 146 to a position corresponding to the desired treatment intensity and pattern. Actuator 145 may be controlled in real-time by controller 200 to modify the light beam so that the microscopic treatment is delivered from handpiece 140 in a uniform or non-uniform pattern across target area 150. Alternately, handpiece 140 may not include a deflector 146. In some embodiments, deflectors, scanners and actuators may be outside of the handpiece (e.g., in the console). Beam outlet end 134 may enter from the top.

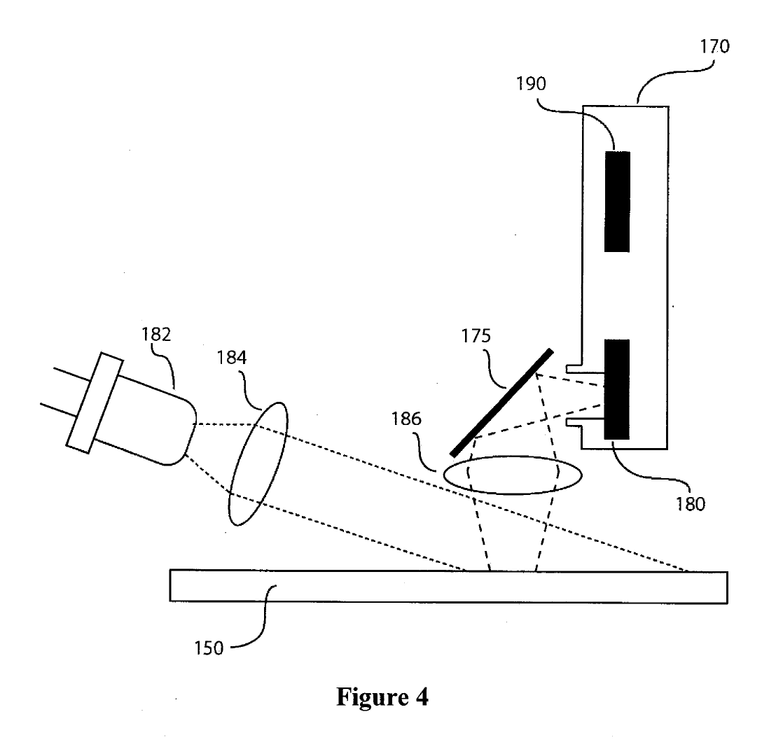

Referring to FIGS. 2-4, handpiece 140 advantageously includes detector 170 for detecting variations in the positional parameters of handpiece 140. Like elements in FIGS. 2-4 have similar reference numerals. Detector 170 may comprise an image acquiring sensor 180 for repeatedly capturing images of target area 150 and image processing device 190 for analyzing in real-time varying positional parameters of the moving handpiece 140. Sensor 180 may be an optical navigation device that allows quantitative measurement of the movement of handpiece 140. The basic operating principle of the optical navigation technique is shown in FIG. 4. Light-emitting diode 182 illuminates the surface of the tissue underneath handpiece 140. The light may be converged by means of converging lens 184 on the treated surface to be reflected off the microscopic textural features in the target area 150. The converged beam of light scattered from the surface is then refocused by converging lens 186 to form an image on position sensor 180. Sensor 180 continuously takes pictures of the points in the treated area at high speed as handpiece 140 moves. The image capturing speed of sensor 180 is sufficiently high to allow sequential pictures to overlap. Sequential images from the sensor 180 are sent to image processing device 190. The optical path of sensor 180 between the target area and the converging lens 186 may include an optically transparent window 155. Image processing device 190 may be a programmable digital computer that uses optical navigation engine for analyzing the sequential images captured by sensor 180.

Possible outcomes from controller 200 can include triggering an “operation” mode and a “stop” mode. In the “operation” mode, the treatment continues, as will be discussed in greater detail below, and the operational parameters of the treatment system 100 are monitored in real-time in response to the signals indicative of the changes in the handpiece positional parameters and/or in response to signals from the tip. In the “stop” mode, controller 200 immediately halts all operations of system 100 in response to detecting that a tip usage parameter has been exceeded or a significant change in treatment conditions that render the continuation of treatment unsafe or ineffective. Specifically, treatment with a dosage level that exceeds a lower threshold for tip usage, for example, but is below the upper threshold is considered acceptable. Treatment at a dosage level that exceeds the upper threshold or is below the lower threshold level may require shutdown of treatment system 100.

A specific example of detector 170 usable in the treatment system 100 is an optical navigation sensor produced by Agilent Technologies, Inc., of Palo Alto, Calif., and particularly the ADNS 2600 series optical navigation engine. The optical navigation engine (i.e. image processing device 190) produces measurements of changes in the handpiece position by optically acquiring sequential surface images up to about 1500 times per second and mathematically determining the direction and magnitude of the handpiece movement at the maximum of 400 counts per inch (cpi) and at speeds up to 12 inches per second (ips).

If an optical navigation sensor such as described in the previous paragraph is used for detector 170, then in some cases this detector can be made more robust by the addition of contrast-enhancing substances, such as particles, dyes, solutions, colloids or suspensions to the target area 150 to enhance the contrast for the optical navigation sensor. One example of contrast enhancing particles would be ink particles that are spread onto the skin by painting or marking the skin prior to treatment with the handpiece. Food dyes may alternately be used for contrast enhancement. Often the contrast-enhancing substance is chosen as an absorber or a reflector of the LED light (e.g., a blue dye may be chosen for use with a red LED).

Returning to FIG. 2, treatment system 100 advantageously includes controller 200 for adjusting in real-time the range of operational parameters of the light beam in response to detected variations in the handpiece positional parameters and/or tip usage and tip parameter information. Controller 200 may be a general purpose programmable digital computer connected to detector 170 and/or tip memory (not shown) to receive a precise digital output. Controller 200 can be programmed to sample in real-time variations in the handpiece positional parameters, tip usage, tip sensor readings, and/or tip parameter information; to display the positional parameters measurements on the display monitor (not shown); to store the measurements; to apply treatment criteria logic to the measured signals and tip information for determining necessary adjustments in operational parameters, and to implement adjustments to at least one operational parameter while the treatment continues. Possible criteria for treatment logic may include changes in the position or the velocity of the handpiece relative to the target area 150, changes in angle of the handpiece relative to the target area 150, changes in the distance of the handpiece from the target area 150, tip usage limits, tip sensor readings, system operational limits imposed by tip type, tip treatment parameter limits and/or tip usage, or combinations thereof. In some embodiments, separate controllers and processors may be used to control and communicate with the tip and the positional controls for the treatment system.

Controller 200 may comprise interface unit 210 for receiving and processing signals indicative of the variations in the positional parameters from detector 170 and/or tip information and sensor readings, analyzing the signals, sending signals requesting determination of suitable operational parameters; and performing adjustments to the signals indicative of operational parameters. Interface unit 210 may include analog processing circuitry (not shown) for normalization or amplification of the signals from detector 170 and an analog to digital converter (not shown) for conversion analog signals to digital signals. Interface unit 210 may be operably coupled to the components of system 100, i.e., power source 110, light emitter 120, actuator 145, tip memory, tip sensors, and tip security chip for selecting initial operational parameters for the tissue treatment and for controllably adjusting in real-time components of the treatment system 100 to generate new suitable operational parameters.

Controller 200 may further include processor 202 for determining a set of desired operational parameters in response to the signals from interface unit 210 indicative of the changes in tip usage or treatment dosage, as well as in response to information on tip type, tip operational parameters, etc. Processor 202 may be embodied as a microprocessor, an ASIC, DSP, or other processing means that are suitable for determining the desired operational parameters. Upon receiving the signals from interface 110, processor 202 determines a new set of suitable operational parameters. Examples of operational parameters for the light emitter 120 are optical power, pulse repetition rate, pulse energy, pulse duty cycle, and wavelength. Examples of other operational parameters are handpiece temperature, actuator 145 movement rate, and actuator 145 movement pattern. Processor 202 may include computational means (not shown) for calculating specific operational parameters, or may be based on neural networks and fuzzy logic techniques for systematically arriving at optimal operational parameters for the desired treatment using the software of this invention. Alternatively, the computational means may comprise a memory look-up tables for generating operational parameters values for a pre-selected treatment given the measured positional parameters, the treatment dosage, tip usage, etc. Memory look-up tables would provide coherent data sets of signal values from detector 170 and corresponding values of desirable operational parameters. Thus, the software of the invention associated with controller 200 allows processor 202 to perform in real-time mapping of operational parameters of treatment system 100 as a function of the handpiece positional parameters, tip data and to output the set of the desired operational parameters to interface unit 110.

Embodiments of the present invention include interchangeable tips on the treatment end of the handpiece. Such tips typically include one or more optical elements 160, LED(s) 182 and window(s) 155. However, various sets of these elements may be included or excluded from the tip. The elements shown in FIGS. 2-4 that are not included in a tip are typically included in the handpiece. Alternate embodiments may include multiple LEDs producing either the same or different wavelengths. Alternate embodiments may further include multiple windows 155 or no windows. In the latter case, an open aperture may replace the window 155.

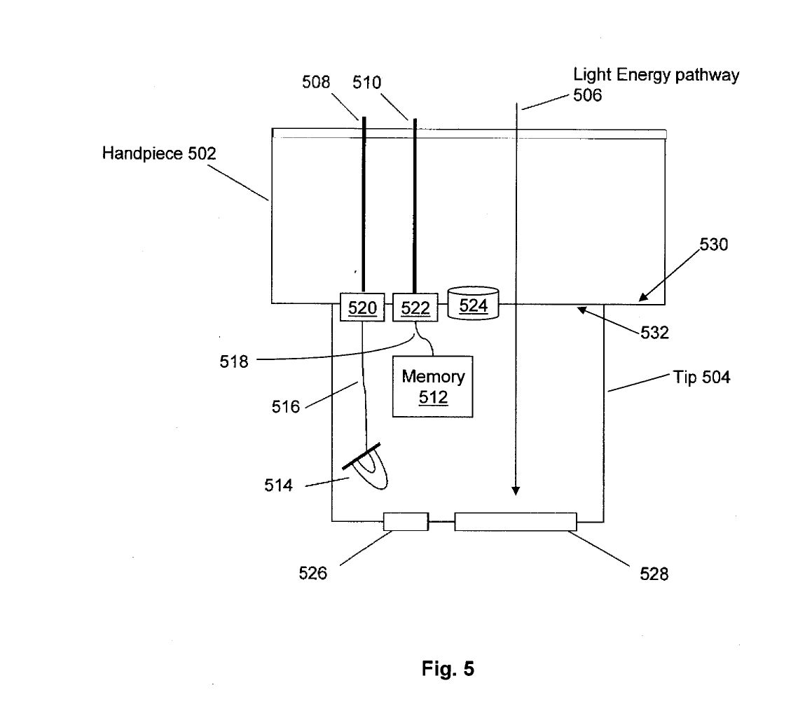

FIG. 5 shows an embodiment of the present invention in which the distal end 530 (i.e., treatment end) of a handpiece 502 is coupled to the proximal end 532 of a tip 504. Handpiece 502 includes one or more electrical connections (e.g., 520, 522) to tip 504. The electrical connection(s) at the interface between tip 504 and handpiece 502 are not permanent, but rather are configured so that when tip 504 is mechanically attached to handpiece 502 the electrical connections are reliably created and maintained during use and treatment. For example, contact pads, spring contacts, pogo pins, ball contacts and the like may be used for electrical contacts (520, 522). Tip 504 is configured to have electrical contacts to match those of the handpiece, although tip 504 may have more or less electrical contacts than a given handpiece. The electrical connections create a communications path between tip 504 and handpiece 502. Handpiece 502 typically has a light energy pathway 506, including, for example, one or more optical fibers, lenses, reflective elements, or diffractive or holographic elements. The light energy pathway in tip 504 may include optics to receive and transmit light energy from the handpiece to a target area of tissue to be treated. Tip 504 may include an aperture 528 through which optical energy from the handpiece passes. In the example shown in FIG. 5, aperture 528 is an open aperture.

Tip 504 typically includes a memory 512 that is attached, either directly or indirectly, to tip 504. Memory 512 may be an EPROM or EEPROM, for example. Memory 512 may be part of a security chip, a control chip, or a microprocessor. Alternately, memory 512 may be a separate and stand alone memory element. For the purposes of this application, a processor may be, for example, a security chip, a control chip, or a microprocessor. Memory 512 is typically connected via one or more wires 518 to an electrical contact to the handpiece 502 in order to communicate with the handpiece 502 and/or a console or system (not shown). Memory 512 may serve various purposes, including tip-system security. Tip-system security typically consists of the memory holding a secure and often encrypted code (e.g., a hash algorithm, for example a 128-bit Secure Hash Algorithm-1 (SHA-1) codes) for use in authenticating the tip to the handpiece and/or treatment system. Typically, the handpiece and/or treatment system includes a controller and a memory holding a similar or matching code to that stored in the tip memory 512. One or more encryption algorithms, handshake protocols and authentication procedures may be used to ensure that an appropriate and specified tip is used with the system. For example, a Dallas Semiconductor DS2432 1 k-Bit Protected 1-Wire™ EEPROM (manufactured by Dallas Semiconductor of Sunnyvale, Calif.) may be used for such secure and encrypted memory. In this example, a single wire may be used between the memory 512 and the system controller and communication may be completed by a 1-wire protocol (e.g., 1-wire SHA-1 protocols).

In one embodiment, the system uses a challenge-response protocol, a keyed hash algorithm, and a secret key to authenticate the tip. The host generates a challenge, such as a random number. The challenge is electronically communicated to the tip. The tip and the host separately concatenate the challenge with their secret keys and generate a hash of that string. The tip sends the hash that it generated back to the host as the response to the challenge. The host compares the response from the tip to the hash that the host created from the challenge. If the two hashes match, then the two secret keys must be identical and the tip is known to be authentic, which enables delivery of light energy to the treatment area. If authentication is not successful, then delivery of light energy to the tip and the treatment area is prevented.

Examples of hash algorithms are MD5, MD4, and SHA1. Those skilled in the art will be able to substitute other hash algorithms. The secret key may be coded into software, stored in memory, or written into a non-readable memory for use by a specialized encryption chip implementing the above protocol. One example of a non-readable memory is sold by Dallas Semiconductor, for example, model numbers DS1963S and DS2432. The method of communication between the tip and the host may be any form of communication, such as the 1-wire protocol, an RF data link, or ethernet. In one embodiment, the DS1963S is used as the host and the DS2432 is used in the tip.

In some embodiments of the present invention, tip memory 512 plays a role in tip usage monitoring and control. In such embodiments, tip memory 512 stores data about tip usage. Such tip usage data may include one or more of the following: number of energy pulses transmitted through the tip; number of pulses emitted by one or more attached light sources; accumulated energy or fluence transmitted through the tip; accumulated energy or fluence emitted by one or more attached light sources; number of treatment zones or spots transmitted to the tissue being treated; area of tissue treated using the tip; power transmitted through the tip and/or transmitted by the one or more attached light sources. A usage limit based on any of the above-listed categories of tip usage data may be stored in tip memory such that when the usage limit is exceeded, the tip and/or the system cease to function in part or in whole. Additionally, other tip usage parameters gathered during treatment with the tip attached may be stored in the memory, such as, for example: pulse repetition rate; wavelength(s); number of sources used; temperature of the tip, handpiece and/or system; number of patients treated; types of treatment regimens used (for example, multiple pass treatments or single pass treatments); etc. These other tip usage parameters may be useful in determining tip life and/or for adjusting treatment parameters over the life of the tip, for example. If the tip is removed from a system and later coupled to the same or a different system, these saved parameters may be useful in monitoring total tip life and/or in setting appropriate treatment parameters taking into account the history of the tip. The handpiece or system to which a tip is attached can access the memory 512 through one or more electrical contacts. A controller or microprocessor in the handpiece or console may read from and/or write to the memory via single or multiple electrical or optical connections using various communication protocols.

An additional layer of security can be provided by embodiments that use an EPROM or EEPROM which has states to which selected data can only be written once. For example, the Dallas Semiconductor DS2432 contains an EPROM that comprises registers that are physically destroyed during the writing of a binary value (e.g. binary value of 1). The binary value is still readable from a bit following the writing step, but the value of that bit cannot be changed. Using the additional layer of security provided by the write-once memory makes it more difficult for someone to utilize a reprogrammed host system to alter the memory on a tip to represent an inaccurate amount of usage. This type of write-once memory is therefore particularly useful in embodiments where tip usage is monitored because it prevents tampering with the usage data to extend the usage life of a tip, which could endanger the patient by allowing treatment with a worn out or damaged tip.

Alternate embodiments may include storing in tip memory a code that unlocks a memory in the treatment system. The unlocked portion of treatment system memory may store information regarding the specific tip-system combination. For example, a tip may store a code relating to a patient or a prior treatment. When such tip is attached to the treatment system and the tip memory is read, the code in tip memory unlocks a section of system memory and patient information or prior treatment parameters are retrieved for use in the current treatment.

In further embodiments of the present invention, tip memory 512 stores tip configuration information, such as, for example: tip width (e.g., tip treatment zone width and/or length); tip focal properties, such as focal length (in air or in tissue) and spot size (typically measured at the tissue surface); tip shape; tip parameter limits; tip treatment parameters; and so forth. Tip shape may include, for example, cross-sectional (i.e. at the treatment end of the tip in a plane perpendicular to the optical axis of the treatment beam and/or parallel to the tissue surface) shapes (e.g., round, oval, polygonal, symmetrical or asymmetrical) or profile (i.e. looking at the tip in a direction substantially perpendicular to the optical axis of a treatment beam transmitted through the tip) shapes typically on the treatment facing side(s) of the tip (e.g., flat faced, rounded, polygonal, indented, bumped, etc.).

Tip shape may also be designed to fit particular anatomical areas, such as for example, small or difficult to reach areas around an eye or a nose. Tip parameter limits may define system parameters within which the tip may safely and/or effectively be used. Such parameter limits may include, for example: energy limits, wavelength(s), pulse repetition rates, power, temperature limits, contacting versus non-contacting treatments, accumulated time of treatment, and so forth. Tip treatment parameters may define treatment parameters to be used when employing a particular tip. For example, tip treatment parameters may include data requiring that a tip be used only around eyes or noses, for example, or that such a tip is particularly suited for treating a specific disease or tissue condition, such as, for example, pigmented lesions or acne. Alternately, tip treatment parameters may indicate that a particular dye or contrast agent be used to enhance the sensing response from an LED within the tip.

A controller or microprocessor in the handpiece and/or in a system console reads the tip configuration information, tip usage information, tip usage parameters and/or other system information from the tip memory and uses software and/or firmware algorithms to create one or more control signal(s) to alter the operation and/or configuration of the system and/or handpiece. Further, the controller and/or microprocessor may cause signals to be sent to an interface unit to provide information to a user. The user can then make treatment decisions or alter the system parameters based thereon, for example, through a touch screen, a keyboard, a mouse or other input mechanism. For example, a tip may store information indicating that it is to be used only for treatments around the eyes, at wavelengths between about 1400 nm and about 1600 nm and at energies less than about 10 mJ. A controller or microprocessor reading this information may send control signals to one or more lasers to produce wavelengths in the range of 1400-1600 nm and with energies no greater than 10 mJ. A user may be notified via an interface unit, such as a monitor, that the tip is primarily for use around eyes, but the user may be offered the option to manually change treatment parameters such as the wavelength and/or energy, among others.

Tip 504 typically includes an LED 514. LED 514 is typically used to illuminate a treatment surface, for example, for targeting purposes or to assist in sensing movement or location of the handpiece relative to the tissue. LED 514 may be coupled via a wire 516 to an electrical connector 520 in order to receive power and/or control signals from the handpiece 502 or a system coupled to the handpiece. Alternately, LED 514 may be attached to a battery within the tip. A given tip 504 may include multiple LEDs. Typically, LED 514 is mounted in tip 504 in an orientation allowing a portion of the light emitted by the LED to pass through an LED aperture 526. In alternate embodiments, LED light may pass through the same aperture as the treatment beam (i.e. aperture 528).

Tip 504 includes a connector mechanism for attaching the tip to a handpiece 502. The proximal end 532 of tip 504 includes a connector mechanism configured to hold the tip in place against the distal end 530 of a handpiece 502 with sufficient force to maintain an electrical contact and an optical coupling between the tip and the handpiece, especially while the handpiece is moved across tissue during treatment. The connector mechanism may take various forms. In the example of FIG. 5, a magnetic connector 524 is shown. Magnets may be placed on one or both sides of the interface between tip 504 and handpiece 502. Alternate attachment mechanisms may include, for example: a clip; a screw; a screw-on connection (i.e. the tip and the handpiece have corresponding male and female threaded portions); an adhesive; a bayonet-style connector; a snap and/or a latch. Additionally, tip 504 is shaped so that it can be removably attached to the distal end 530 of handpiece 502. Dowels and corresponding holes may be used to seat and hold the tip against the handpiece. Further, the shape of the tip may be configured to fit the handpiece tightly. For example, the tip shape may be more conformal to match the shape of the distal end of the handpiece, rather than being a flat surface that butts flush against a flat surface of the handpiece.

Tip 504 may further include one or more sensors (not shown) for monitoring various parameters of the tip, treatment beam and/or the tissue being treated. For example, a monitor photodiode may be included in the tip to monitor the treatment beam. This may require a partially reflective element to monitor a portion of the treatment beam. This real-time monitoring of treatment beam characteristics may be used to alter the system and/or treatment parameters. As a further example, a temperature sensor, such as, for example, a thermocouple may be coupled to the tip. In some embodiments a thermocouple is attached to the tip at or near the treatment end of the tip, so as to monitor tissue surface temperature. Such sensors are typically in communication with the treatment system, either electrically, optically or by wireless connection. Further, some embodiments may include radio-frequency identification (RF ID) chips as a further security measure (i.e. if a RF ID communication system is included in the system to check the RF ID on the tip) and for tracking purposes to identify individual chips and their locations. Such RF ID chips may store some of the data and codes described above as stored in the tip memory.

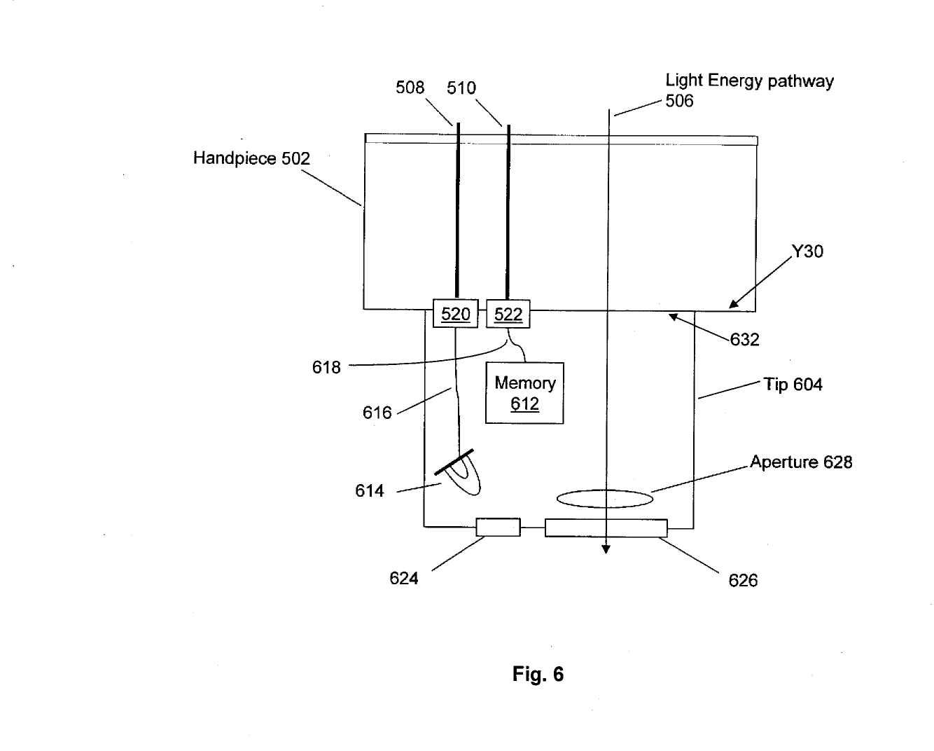

FIG. 6 is an alternate embodiment of the present invention. FIG. 6 is similar to FIG. 5 in that the same handpiece components and handpiece configuration are depicted in FIG. 6. As such, in FIG. 6 like elements are numbered similarly to those in FIG. 5. Tip 604 differs from tip 504 in FIG. 5 at least in that tip 604 has a different connector mechanism (i.e. it does not show a magnet) and tip 604 has window(s) 626, 624 and an aperture 628. The memory 612, LED 614, and electrical connections 616, 618 are similar to corresponding parts in FIG. 5 and serve similar functions.

Window 624 is a window through which light emitted by LED 614 is transmitted. Window 626 is a treatment window through which light emitted from the system (i.e. from handpiece 502) is transmitted to a tissue surface. Window 624 and treatment window 626 may be a single window in some embodiments. Windows 624 and 626 are typically made of glass, sapphire, diamond, quartz or silica, although other substances may be chosen for their optical and/or thermal properties. In some embodiments, windows 624 and/or 626 may include filters for limiting the transmission of one or more wavelengths. For example, in systems having multiple lasers or light sources emitting multiple wavelengths, a tip may be chosen to transmit or block one or more wavelengths depending on the desired treatment parameters. Such filters may include thin film filters, reflectors and/or coatings in single-layer or multi-layer configurations. Such filters may be absorptive or reflective or a combination thereof. Such filters may include doped glass filters, fused silica with a dielectric coating, silicon, etc. Alternately, window 626 may include diffractive, holographic, polarizing elements, opto-electronic elements, acousto-optic elements, a lens, an optical limiter, a saturable absorber, or a passive q-switch element to alter the light transmitted through the window and/or to alter the treatment pattern and spot dimensions on the tissue.

Aperture 628 is optional. Aperture 628 may be used to limit the numerical aperture of the system and/or to limit the size of the treatment pattern at the tissue. For example, if the handpiece produces a set number of spots (e.g. 30 across a single line perpendicular to the direction of movement of the handpiece) in a given treatment pattern to create a set treatment zone dimension (i.e. 15 mm wide), then aperture 628 may be used with a smaller dimensioned tip to limit the treatment to fewer spots and a narrower treatment zone (e.g., 15 spots across an 8-mm-wide line). Aperture 628 may include a reflective coating to direct light incident thereon in a desired direction, such as, for example, to a beam dump or an absorbing heat sink. Alternately, aperture 628 may be a heat sink or an absorber.

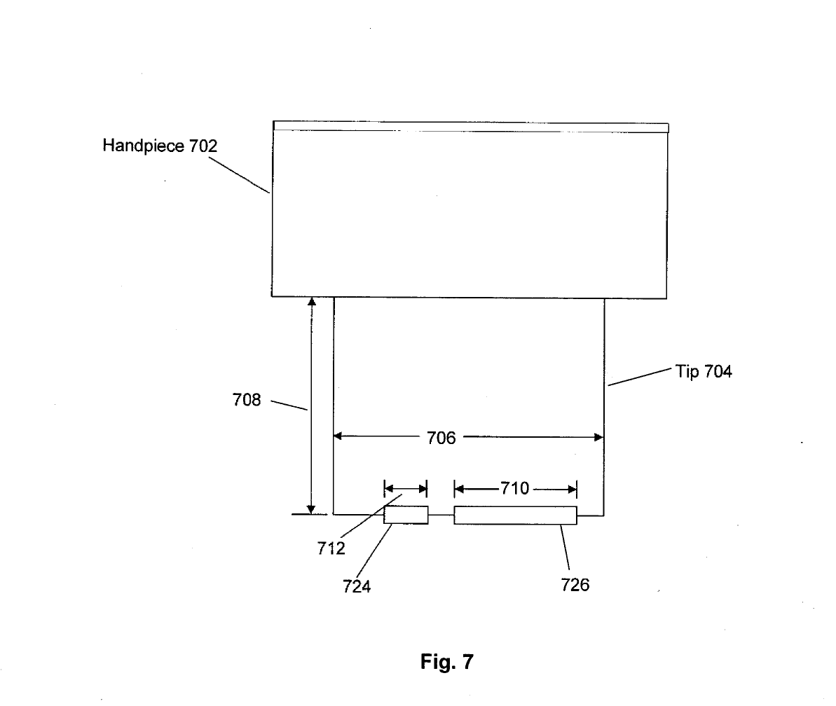

FIG. 7 shows an example of the various tip 704 dimensions that may be changed between individual tips that are interchangeable with the same handpiece 702. A set of two or more tips are configured to fit and be attached to a given handpiece. Each individual tip in the set is different in at least one aspect from the other individual tips in the set. The differences between tips may include differences in: dimensions; shapes; windows; filters; memory configurations or sizes; number and type of LED(s); aperture(s); additional connector mechanisms; number and type of electrical connections; additional sensors; security codes, operational parameters and/or other information stored in tip memory; and so forth. FIG. 7 shows some of the dimensions that may be different between individual tips in a set of tips. For example, the width 706 may be different from one tip to the next. Additionally, window width(s) 712 and/or 710 may be different from one tip to the next. Altering treatment window width 710 may also impact the aperture of tip for treatment purposes. The length 708 may also be different from one tip to the next. Length 708 will typically alter the focal depth of a treatment beam relative to the treatment window 726 and the surface of tissue being treated, as well as the spot size of the treatment beam at the tissue surface.

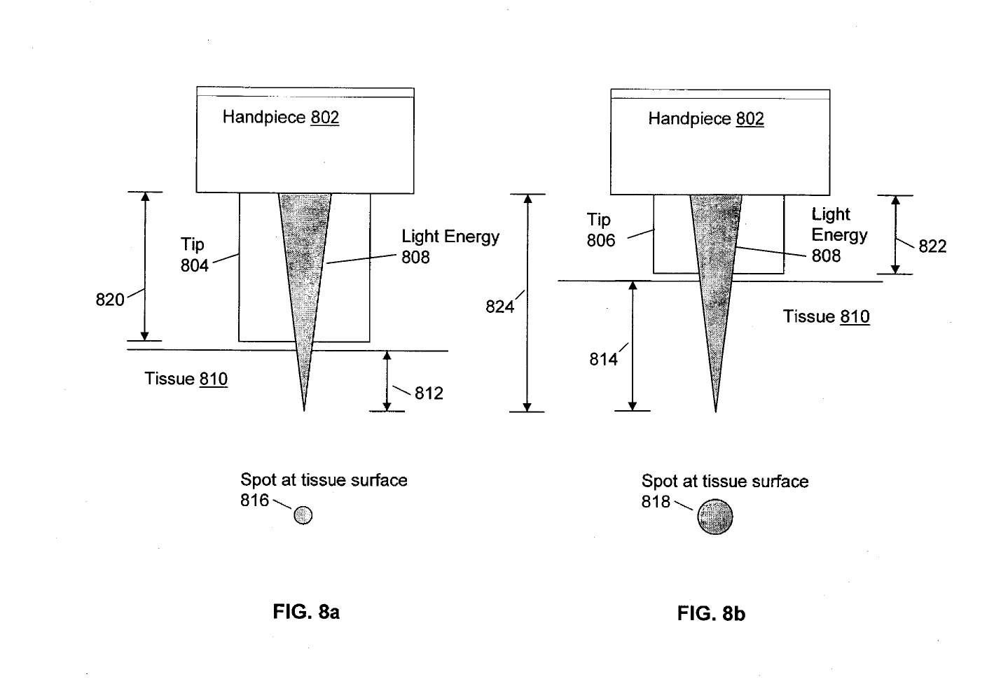

FIGS. 8a and 8b illustrate the effects of altering a length dimension for two tips (804 and 806) each attached separately at different times to the same treatment system and handpiece 802. In FIGS. 8a and 8b, like elements are referenced with like reference numerals. A handpiece (e.g., in the absence of a tip) produces a treatment beam having a set focal length 824, numerical aperture and beam profile (i.e. light energy beam 808). Tip 804 has a different length 820 from the length 822 of tip 806. This difference in tip length will alter the depth in the tissue (i.e. depth 812 for tip 804 vs. depth 814 for tip 806) at which the beam will focus (i.e. the beam waist) and will also impact the spot size at the tissue surface (i.e. spot 816 for tip 804 vs. spot 818 for tip 806). Altering spot size, spot shape and/or focal depth from one tip to another significantly impacts the treatment parameters and typically the tissue response. For example, a larger spot size may cause a larger treatment zone lesion, and a deeper focal point may cause a deeper treatment zone lesion and/or necrotic zone. Further, altering these treatment dimensions may alter the shape and size of the treatment zone.

The foregoing describes a system and method for laser surgery wherein a focused optical signal such as a laser, LED, or an incoherent source of optical energy is advantageously created to achieve treatment zones using interchangeable tips. Persons of ordinary skill in the art may modify the particular embodiments described herein without undue experimentation or without departing from the spirit or scope of the present invention. All such departures or deviations should be construed to be within the scope of the following claims.

1. A medical electromagnetic energy treatment system comprising:
a removable tip apparatus comprising:
a housing shaped to be removably attachable to a treatment end of the medical electromagnetic energy treatment system;
a first memory attached to the housing, containing a first secure code, and containing electronically readable states that can only be written once; and
a communications device attached to the housing, for establishing communication with the medical electromagnetic energy treatment system for authenticating the tip apparatus based on the first secure code when the housing is attached to the treatment end of the medical electromagnetic energy treatment system.
